(19) United States
(12) Patent Application Publication
(10) Pub. No.: US 2023/0248404 A1
(43) Pub. Date: Aug. 10, 2023

(54) BONE PLATE AND BONE FRACTURE TREATMENT KIT

(71) Applicant: MEIRA Corporation, Nagoya-shi (JP)

(72) Inventors: Kazuki TANAHASHI, Minokamo-shi (JP); Kazuya YOGO, Seki-shi (JP)

(73) Assignee: MEIRA Corporation, Nagoya-shi (JP)

(21) Appl. No.: 18/163,978

(22) Filed: Feb. 3, 2023

(30) Foreign Application Priority Data
Feb. 8, 2022 (JP) 2022-018263

(51) Int. Cl. A61B 17/80; A61B 17/88

(52) U.S. Cl. CPC A61B 17/8061; A61B 17/8052; A61B 17/8014; A61B 17/8863

(57) ABSTRACT

A bone plate includes a plate body portion extending in a lengthwise direction, a head portion extending from one end portion in the lengthwise direction of the plate body portion, and tabs provided on the outer side of an outer peripheral portion of the head portion. The tabs comprise first-type tabs and side by side with each other, and a second-type tab located on one outside of the first-type tabs. The first-type tabs respectively include first connecting portions that are bendable and have a cross-sectional area in a widthwise cross section that is smaller than the cross-sectional area of the outer shape of the first tab body portions. The second-type tab includes a second connecting portion that is bendable and twistable and has a smaller cross-sectional area in a widthwise cross section than the first connecting portions.

### TECHNICAL FIELD

The present invention relates to a bone plate used for treatment of a fracture in an end portion of a long bone and a bone fracture treatment kit.

### BACKGROUND ART

Some human bones have an elongated shape (a longer length than width), and such bones are sometimes called long bones. Such long bones have a diaphysis that makes up the central portion, epiphyses that make up the two end portions, and metaphyses that make up the portions between the diaphysis and the epiphyses.

For example, there are two long bones in the portion of each lower leg from the knee to the ankle. The thinner one on the outward side is the fibula, and the thicker one on the inward side is the tibia. The tibia forms the ankle joint together with the fibula and the talus at the end portion near the foot (i.e., the distal portion). If a strong external force is applied to the ankle joint, a fracture may appear in the distal portion of the tibia near the ankle joint. A fracture in the distal portion of the tibia may produce a large number of fracture fragments that require rejoining.

When treating a fracture in an end portion of a long bone, including a fracture in the distal portion of the tibia, the positions and the postures of fracture fragments are first corrected, and then the fracture fragments and the bone main body are fixed, and this is performed with use of a bone plate that is attached as a bridge between the bone main body and the fracture fragments.

As one example of a bone plate for treatment of fracture in the distal portion of the tibia, JP 5507463B discloses an anterolateral plate for the anterolateral face of the distal tibia. JP 5507463B discloses an anterolateral plate for the anterolateral face of the distal tibia, and this anterolateral plate includes a peduncle that twists about the lengthwise axis, a distal head portion that extends from only one side of the peduncle, widens relative to the peduncle, and transitions to a lateral extension portion, and a plurality of distal tabs that are connected to the distal end of the distal head portion, each of the distal tabs including a threaded hole and being connected to the distal end of the head portion by a deformable bridge.

### PRIOR ART DOCUMENTS

### Patent Documents

Patent Document 1: JP 5507463B (WO2009/073405A2, US2009-143825A, US2013-006312A, EP2227160A, EP2756814A)

### SUMMARY OF THE INVENTION

The shapes of bones differ from patient to patient, and the condition of a fracture (e.g., the positions and postures of fracture fragments) also differs from case to case. For this reason, in clinical practice for example, a bone plate manufactured with a predetermined shape in advance may be deformed by a doctor to conform to the shape of a certain bone and the condition of the fracture. For example, in the above-mentioned treatment of a fracture in the distal portion of the tibia, in order to mitigate ankle joint dysfunction and discomfort after healing even a little, there is demand for a bone plate that can treat the fracture more appropriately (correct the positions and postures of the fracture fragments and maintain the corrected state), or in other words, a bone plate that can be more freely deformed according to the shape of the bone and the condition of the fracture.

In view of this, an object of the present invention is to provide a bone plate and a bone fracture treatment kit that are for use in treatment of a fracture in the end portion of a long bone, can be more freely deformed according to the shape of the bone and the condition of the fracture, and allows more appropriately treating a fracture in the end portion of a long bone.

The following is an aspect of the present invention for achieving the aforementioned objective.

A bone plate for use in treatment of a fracture in an end portion of a long bone, comprising:

a plate body portion extending in a lengthwise direction;

a head portion extending from one end portion in the lengthwise direction of the plate body portion and having a larger width than the one end portion; and

tabs on an outer side of an outer peripheral portion of the head portion, wherein

the head portion has a plurality of head portion through-holes into which a shaft portion of a bone screw is insertable,

the tabs comprise first-type tabs side by side with each other on the outer side of the outer peripheral portion of the head portion and a second-type tab on one outside of the first-type tabs,

each of the first-type tabs includes:
a first tab body portion; and
a first connecting portion that is rod-shaped or plate-shaped and connects the first tab body portion and the outer peripheral portion of the head portion,

a cross-sectional area of a widthwise cross section of the first connecting portion is smaller than a cross-sectional area of a widthwise cross section of an outer shape of the first tab body portion,

the first connecting portion is bendable,

the second-type tab includes:
a second tab body portion having a second tab through-hole; and
a second connecting portion that is rod-shaped or plate-shaped and connects the second tab body portion and the outer peripheral portion of the head portion,

a minimum cross-sectional area of a widthwise cross section of the second connecting portion is smaller than a minimum cross-sectional area of a widthwise cross section of the first connecting portion,

the second connecting portion is bendable and twistable, and

wherein the bone plate is for use in treatment of a fracture in an end portion of a long bone.

The above object also can be achieved by the following.

A bone plate for use in treatment of a fracture in an end portion of a long bone, comprising:

a plate body portion extending in a lengthwise direction;

a head portion extending from one end portion in the lengthwise direction of the plate body portion and having a larger width than the one end portion; and

tabs on an outer side of an outer peripheral portion of the head portion, wherein

the tabs comprise first-type tabs side by side with each other on the outer side of the outer peripheral portion of the head portion, a second-type tab on one outside of the first-type tabs; and a third-type tab on an outside of the first-type tabs opposite to a side on which the second-type tab is provided,

each of the first-type tabs includes:
a first tab body portion; and
a first connecting portion that is rod-shaped or plate-shaped and connects the first tab body portion and the outer peripheral portion of the head portion,

a cross-sectional area of a widthwise cross section of the first connecting portion is smaller than a cross-sectional area of a widthwise cross section of an outer shape of the first tab body portion,

the first connecting portion is bendable,

the second-type tab includes:
a second tab body portion having a second tab through-hole; and
a second connecting portion that is rod-shaped or plate-shaped and connects the second tab body portion and the outer peripheral portion of the head portion,

a minimum cross-sectional area of a widthwise cross section of the second connecting portion is smaller than a minimum cross-sectional area of a widthwise cross section of the first connecting portion,

the second connecting portion is bendable and twistable,

the third-type tab includes:
a third tab body portion having a third tab through-hole; and
a third connecting portion that is rod-shaped or plate-shaped and connects the third tab body portion and the outer peripheral portion of the head portion,

due to the third connecting portion, the third-type tab extends outward from the head portion in a different direction from the first-type tabs,

a minimum cross-sectional area of a widthwise cross section of the third connecting portion is larger than the minimum cross-sectional area of the widthwise cross section of the second connecting portion, and

the third connecting portion is bendable.

The above object also can be achieved by the following.

A bone fracture treatment kit comprising:

the bone plate and a deformation assisting member for the bone plate,

wherein the bone plate includes deformable portions having an opening extending through thereof,

the deformable portions include the first-type tabs and/or the second-type tab,

the openings form first tab through-holes provided in the first tab body portions of the first-type tabs and/or the second tab through-hole of the second-type tab,

the deformation assisting member includes an assisting member main body having a thickness in a vertical direction,

the assisting member main body has a lower surface including a plate contact portion configured to come into contact with the bone plate,

the assisting member main body includes:
a plate facing portion configured to be located a predetermined distance above the openings of the deformable portion of the bone plate when the deformation assisting member is overlaid on the bone plate and the plate contact portion is in contact with the bone plate; and
a deforming tool insertion hole that penetrates through the plate facing portion and has a hard inner peripheral surface, and through which a rod-shaped deforming tool is insertable,

the deformation assisting member is further configured such that the deforming tool can be inserted into the deforming tool insertion hole and the opening of the deformable portion of the bone plate simultaneously, and the deforming tool is displaceable in the inserted state, and

an amount of deformation of the deformable portion of the bone plate is restricted by the deforming tool coming into contact with the hard inner peripheral surface of the deforming tool insertion hole.

### BRIEF DESCRIPTION OF THE DRAWINGS

FIG. 1 is a front view showing an embodiment of a bone plate according to the present invention.

FIG. 2 is a left side view showing the embodiment of the bone plate according to the present invention.

FIG. 3 is a right side view showing the embodiment of the bone plate according to the present invention.

FIG. 4 is a plan view showing the embodiment of the bone plate according to the present invention.

FIG. 5 is a bottom view showing the embodiment of the bone plate according to the present invention.

FIG. 6 is a rear view showing the embodiment of the bone plate according to the present invention.

FIG. 7 is an enlarged illustrative view of a cross-section taken along A-A in FIG. 1.

FIG. 8 is an enlarged illustrative view of a cross-section taken along B-B in FIG. 1.

FIG. 9 is an enlarged illustrative view of a cross-section taken along C-C in FIG. 1.

FIG. 10 is an illustrative view showing an example of the case in which the bone plate shown in FIG. 1 is used at the distal portion of a tibia.

FIG. 11 is a front view showing an example of a bone screw used together with the bone plate shown in FIG. 1.

FIG. 12 is a front view showing another embodiment of the bone plate according to the present invention.

FIG. 13 is a front view showing an embodiment of a deformation assisting member of a bone fracture treatment kit according to the present invention.

FIG. 14 is a plan view showing the embodiment of the deformation assisting member of the bone fracture treatment kit according to the present invention.

FIG. 15 is a left side view showing the embodiment of the deformation assisting member of the bone fracture treatment kit according to the present invention.

FIG. 16 is a right side view showing the embodiment of the deformation assisting member of the bone fracture treatment kit according to the present invention.

FIG. 17 is a rear view showing the embodiment of the deformation assisting member of the bone fracture treatment kit according to the present invention.

FIG. 18 is a bottom view showing the embodiment of the deformation assisting member of the bone fracture treatment kit according to the present invention.

FIG. 19 is an enlarged illustrative view of a cross-section taken along X-X in FIG. 14.

FIG. 20 is a front view showing a deforming tool in which the deformation assisting member shown in FIG. 13 is suitably used.

FIG. 21 is a front view showing a tool main body portion of the deforming tool shown in FIG. 20.

FIG. 22 is an illustrative view showing one step in a deformation process of the bone plate using the bone fracture treatment kit in the present invention.

FIG. 23 is a right side view of FIG. 22.

FIG. 24 is an illustrative view showing one step in the deformation process of the bone plate using the bone fracture treatment kit in the present invention.

FIG. 25 is a right side view of FIG. 24.

### EMBODIMENTS OF THE INVENTION

A bone plate according to the present invention will be described below using the embodiments shown in the drawings.

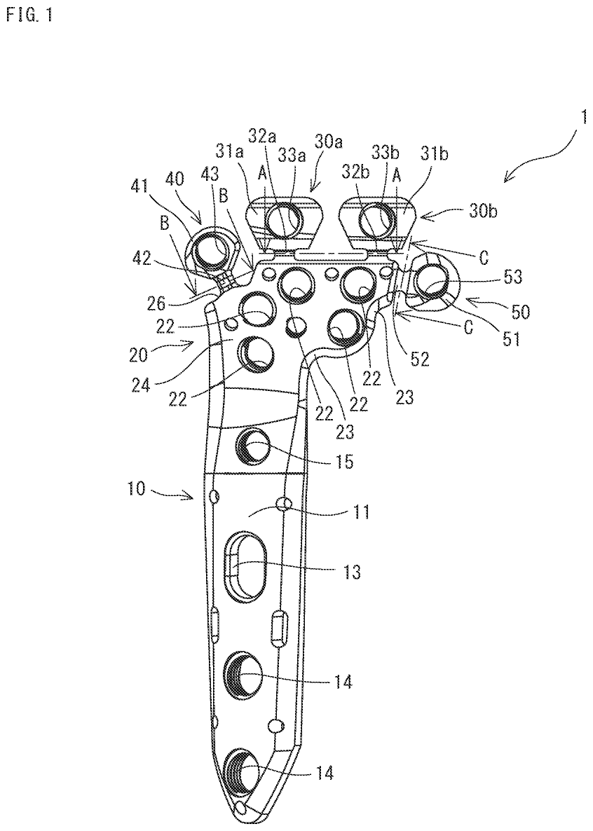

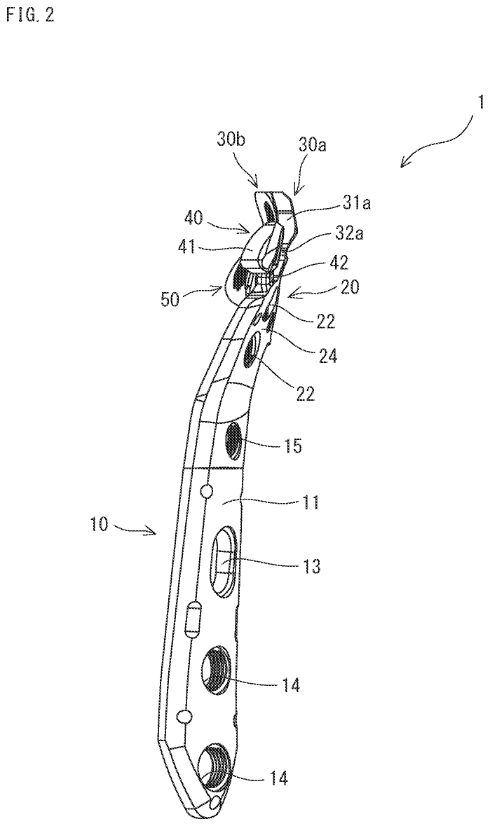

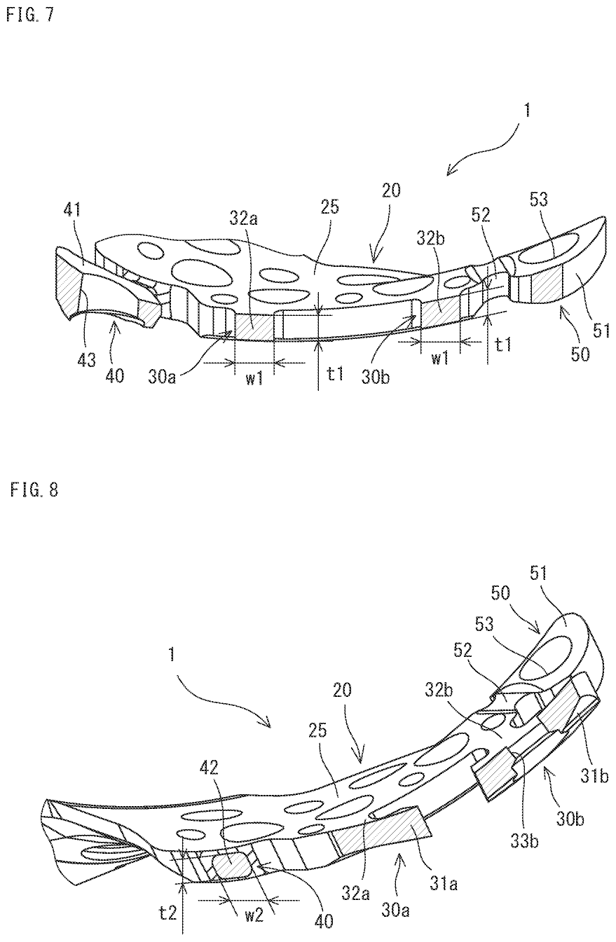

A bone plate 1 of the present invention is a bone plate 1 for use in treatment of a fracture in an end portion of a long bone, and includes a plate body portion 10 extending in a lengthwise direction (up-down direction in FIG. 1), a head portion 20 extending from one end portion in the lengthwise direction (upper end portion in FIG. 1) of the plate body portion 10 and having a larger width (larger dimension in the width direction, which is the left-right direction in FIG. 1) than the one end portion, and tabs (30, 40, 50) provided on an outer side of an outer peripheral portion (on a peripheral edge portion) of the head portion 20. The head portion 20 has a plurality of head portion through-holes 22 into which a shaft portion 71 of a later-described bone screw 7 can be inserted. The tabs (30, 40, 50) include first-type tabs 30 (30a, 30b) provided side by side with each other on the outer side of the outer peripheral portion of the head portion 20, and a second-type tab 40 located on one outside of the first-type tabs 30 (30a,30b) (specifically, located on a left portion of the head portion 20 in FIG. 1). The first-type tabs 30 (30a, 30b) each include a first tab body portion 31 (31a, 31b) and a first connecting portion 32 (32a, 32b) that is rod-shaped or plate-shaped and connects the first tab body portion 31 (31a, 31b) and the outer peripheral portion of the head portion 20, and furthermore, the cross-sectional area of a widthwise cross section of the first connecting portion 32 (the cross section shown in later-described FIG. 7) is smaller than the cross-sectional area of a widthwise cross section of the outer shape of the first tab body portion 31, and the first connecting portion 32 is bendable. The second-type tab 40 includes a second tab body portion 41 having a second tab through-hole 43, and a second connecting portion 42 that is rod-shaped or plate-shaped and connects the second tab body portion 41 and the outer peripheral portion of the head portion 20, and furthermore, the minimum cross-sectional area of a widthwise cross section (the cross section shown in later-described FIG. 8) of the second connecting portion 42 is smaller than the minimum cross-sectional area of a widthwise cross section of the first connecting portion 32, and the second connecting portion 42 is bendable and twistable.

Note that the bone plate 1 shown in the present embodiment is a bone plate for use on the anterolateral face of the distal portion of the tibia (i.e., a tibia anterolateral plate), and is for use with the left leg (the bone plate for use with the right leg is bilaterally symmetrical, except for the spiral direction of the female threading). Also, in the following description, in accordance with how the bone plate 1 is used with respect to the distal portion of the tibia, the direction toward one side in the lengthwise direction will be referred to as the distal direction, and the direction toward the other side in the lengthwise direction will be referred to as the proximal direction.

The plate body portion 10 of the bone plate 1 extends in the lengthwise direction and constitutes the proximal portion of the bone plate 1. The plate body portion 10 is mainly arranged on the diaphyseal portion that constitutes the central portion of the long bone. The plate body portion 10 is twisted about the lengthwise axis, and has an upper surface 11 and a lower surface 12. When the bone plate 1 is used, the lower surface 12 is arranged on the bone side (the side that comes into contact with the bone). The plate body portion 10 of the bone plate 1 is provided with a fixing hole 13 shaped as a rectangle having rounded corners (oval shape) in a plan view. The inner surface of the fixing hole 13 does not have female threading. In a normal procedure, initial fixing of the bone plate 1 to the target site (diaphyseal portion of the long bone) is performed using the fixing hole 13 and a bone screw (not having threading (male threading) on the head portion).

A plurality of (here, two on the proximal side of the fixing hole 13) screw holes 14 for fixing bone screws are provided in the vicinity of the fixing hole 13 of the plate body portion 10. Female threading is provided on the inner surfaces of the screw holes 14. The female threading is formed parallel with the axial direction of the screw holes 14. The female threading can be screwed with male threading provided on the head portions of bone screws (not shown). The bone screws are attached and fixed to the bone plate 1 (plate body portion 10) by being screwed to the female threading. Also, a plate body portion through-hole 15, into which a shaft portion 71 of a later-described bone screw 7 can be inserted (in other words, having a larger diameter than the shaft portion 71), is provided on the distal side of the fixing hole 13 of the plate body portion 10. In the present embodiment, the plate body portion through-hole 15 has the same diameter as a later-described head portion through-holes 22 (i.e., the diameters of the preparatory holes before formation of the female threading are the same). Also, in the present embodiment, female threading is provided on the inner surface of the plate body portion through-hole 15, and the female threading can be engaged or screwed with male threading provided on a head portion 72 of the later-described bone screw 7. Accordingly, the same type of bone screw 7 as that used with the head portion through-holes 22 can be used with the plate body portion through-hole 15. Also, as shown in FIG. 1, the plate body portion 10 has a plurality of small holes into which guide pins (thin, pointed metal pins, which are also called K wires or Kirschner wires) can be inserted, in addition to the fixing hole 13 and the screw holes 14. This enables temporarily fixing the bone plate 1 with use of the guide pins.

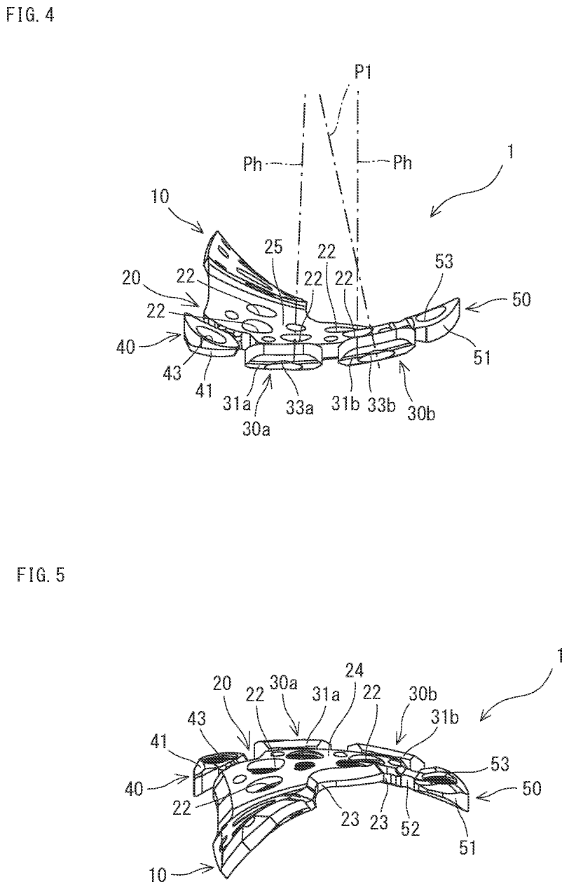

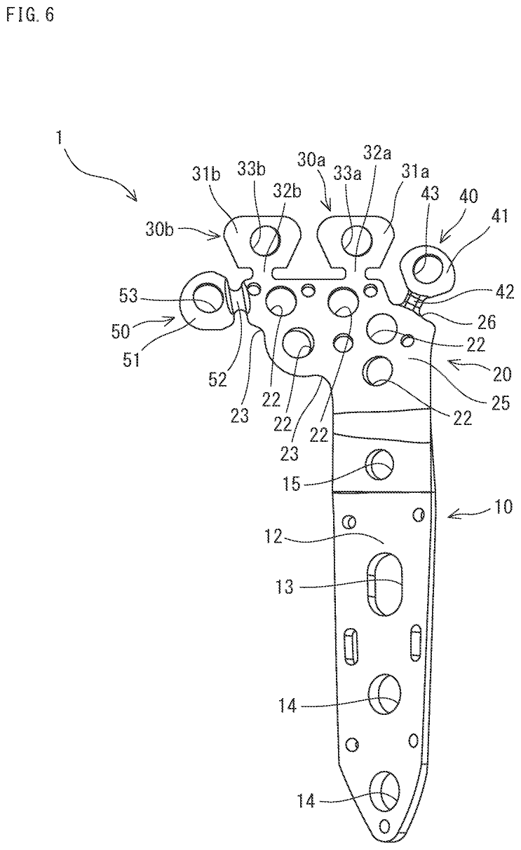

The head portion 20, which is wider than the distal portion of the plate body portion 10, is provided on the distal side of the plate body portion 10. The head portion 20 is mainly arranged at an epiphyseal portion (sometimes including part of the metaphyseal portion) that constitutes the end portion of the long bone. In the present embodiment, the width of the head portion 20 increases stepwise toward the distal direction and one side in the width direction (the right side in FIG. 1). Accordingly, as shown in FIG. 1, two recessed portions 23 are formed in the outer shape of the bone plate 1, thus reducing the region of contact between the bone plate 1 and the skin or the like. The head portion 20 includes an upper surface 24 and a lower surface 25, which are respectively continuous with the upper surface 11 and the lower surface 12 of the plate body portion 10. When the bone plate 1 is used, the lower surface 25 is arranged on the bone side (the side that comes into contact with the bone). As shown in FIG. 4, the head portion 20 is overall curved so as to protrude toward the upper surface (downward in FIG. 4).

In the bone plate 1, the plate body portion 10 and the head portion 20 constitute the base portion of the bone plate 1. In the present embodiment, the lower surfaces (the lower surface 12 and the lower surface 25) of the base portion of the bone plate 1 are overall continuous free curved surfaces (without geometrically shaped portions).

The head portion 20 has a plurality of (here, five) head portion through-holes 22 into which the shaft portion 71 of a later-described bone screw 7 can be inserted (in other words, having a larger diameter than the shaft portion 71). In the present embodiment, the head portion through-holes 22 all have the same diameter (i.e., the diameters of the preparatory holes before formation of the female threading are the same). Also, in the present embodiment, female threading is provided on the inner surface of the head portion through-holes 22, and the female threading can be engaged or screwed with male threading provided on the head portion 72 of the later-described bone screw 7. This allows the same type of bone screw 7 to be applied to the head portion through-holes 22. Also, as shown in FIG. 1, the head portion 20 has a plurality of small holes into which guide pins (thin, pointed metal pins, which are also called K wires or Kirschner wires) can be inserted, in addition to the head portion through-holes 22. Accordingly, by using a guide pin, it is possible to confirm the insertion state (e.g., position, orientation, and insertion amount) of a bone screw 7 inserted into a head portion through-hole 22, and to temporarily fix a fracture fragment.

Tabs (30, 40, 50) are provided on a peripheral edge portion of the head portion 20. In the present embodiment, the tabs (30, 40, 50) include two first-type tabs 30 (hereinafter, in the present embodiment, configurations related to the first-type tab 30 provided on the left side in FIG. 1 will sometimes have “a” appended to the reference number, and configurations related to the first-type tab 30 provided on the right side will sometimes have “b” appended to the reference number), the second-type tab 40, and a third-type tab 50.

The first-type tabs 30a and 30b are provided side by side with each other in the width direction, and extend in the distal direction from the distal end portion of the head portion 20. The first-type tabs 30a and 30b respectively include bendable first connecting portions 32a and 32b, and extend in the distal direction from the distal end portion of the head portion 20 due to the first connecting portions 32a and 32b. Portions of the first-type tabs 30a and 30b on the distal side of the first connecting portions 32a and 32b (i.e., the portions other than the first connecting portions 32a and 32b) are first tab body portions 31a and 31b.

The cross-sectional area of the first connecting portions 32a and 32b is smaller than the cross-sectional area of the outer shape of the first tab body portions 31a and 31b. More specifically, as shown in FIGS. 1 and 7, the first connecting portions 32a and 32b have a smaller width (w1) than the first tab body portions 31a and 31b. In other words, in the first-type tabs 30a and 30b, the rigidity of the first connecting portions 32a and 32b is lower than that of the first tab body portions 31a and 31b. Accordingly, at the first connecting portions 32a and 32b, the first-type tabs 30a and 30b are bendable toward the upper surface and the lower surface of the plate body portion 10 and the head portion 20. Note that the thickness (t1) of the first connecting portions 32a and 32b may be smaller than the thickness of the first tab body portions 31a and 31b.

Note that it is desirable that the first connecting portions 32a and 32b are bendable but not twistable (when a doctor performs an operation, the first connecting portions 32a and 32b substantially cannot be twisted about the extending direction of the first-type tabs 30a and 30b). For example, it is preferable that the width w1 of the first connecting portions 32a and 32b (about 2.4 to 2.6 mm in the present embodiment) is larger than the thickness t1 of the first connecting portions 32a and 32b (about 1.59 to 1.69 mm in the present embodiment), and it is preferable that the ratio of the width w1 to the thickness t1 (w1/t1) is in the range of 1.5 to 2.0. Accordingly, in comparison with the later-described second connecting portion 42, the first connecting portions 32a and 32b are bendable but substantially not twistable.

The first tab body portions 31 (31a, 31b) are located on the distal side of the first-type tabs 30 (on the side toward which the first-type tabs 30 extend). In the present embodiment, as shown in FIG. 1, the first tab body portions 31a and 31b of the first-type tabs 30a and 30b are each substantially shaped as a trapezoid that widens in the distal direction (toward one side in the lengthwise direction, or in other words, toward the epiphysis of the long bone). Accordingly, the fractured portion (particularly, the bone and the fracture fragment at the epiphysis) can be appropriately supported by the first tab body portions 31a and 31b (particularly, the distal portions where the first tab body portions 31a and 31b are wider).

Note that although the first tab body portions 31a and 31b of the first-type tabs 30a and 30b widen at substantially the same angle on both sides in the width direction in the present embodiment, the angle of widening may be different on the two sides in the width direction. Also, in the first tab body portions 31a and 31b of the adjacent first-type tabs 30a and 30b, it is preferable that the side portions that face each other (the right side of the first tab body portion 31a and the left side of the first tab body portion 31b) widen toward the first tab body portions 31b and 31a of the adjacent first-type tabs 30b and 30a. Note that in the first-type tabs 30a and 30b having a substantially trapezoidal shape, it is preferable that the maximum width dimension is 1.5 times or more the minimum width dimension (here, the width dimension of the base end portions (the end portions on the first connecting portion 32a and 32b side) of the first-type tabs 30a and 30b).

The first tab body portions 31a and 31b of the first-type tabs 30a and 30b respectively have first tab through-holes 33a and 33b. In the present embodiment, the first tab through-holes 33a and 33b have the same diameter as the head portion through-holes 22 (i.e., the diameters of the preparatory holes before formation of the female threading are the same). Also, female threading is provided on the inner surfaces of the first tab through-holes 33a and 33b of the first-type tabs 30a and 30b, and the female threading can be engaged or screwed with male threading provided on the head portion 72 of the later-described bone screw 7. Accordingly, the same type of bone screw 7 as that used with the head portion through-holes 22 can be used with the first tab through-holes 33a and 33b.

As described above, the first tab body portions 31a and 31b of the first-type tabs 30a and 30b have the first tab through-holes 33a and 33b. Here, it is preferable that in the projected area of the first tab through-holes 33a and 33b as viewed from the axial direction, the projected area of the outer shape of the first tab body portions 31a and 31b is 4 times or more the projected area of the first tab through-holes 33a and 33b (the area of the preparatory hole). Accordingly, even in the case where the first tab through-holes 33a and 33b are formed in the first tab body portions 31a and 31b, the fractured portion (particularly, the bone and the fracture fragment at the epiphysis) can be appropriately supported by the first tab body portions 31a and 31b.

Specifically, in the present embodiment, the projected area of the outer shape of the first tab body portions 31a and 31b is 55.6 mm², the projected area of the first tab through-holes 33a and 33b is 12.1 mm² (the diameter of the preparatory hole before forming the female threading is 3.93 mm), and the projected area of the outer shape of the first tab body portions 31a and 31b is 4.6 times the projected area of the first tab through-holes 33a and 33b. Note that it is preferable that the projected area of the outer shape of the first tab body portions 31a and 31b is in the range of 4 to 5 times the projected area of the first tab through-holes 33a and 33b (the area of the preparatory hole).

In the bone plate 1 of the present embodiment, two first-type tabs 30 (30a, 30b) are provided, the first tab body portions 31a and 31b of the two first-type tabs 30a and 30b respectively have the first tab through-holes 33a and 33b, and the centers of the two head portion through-holes 22 formed on the distal side (one side in the lengthwise direction) in the head portion 20 are located between the centers of the two first tab through-holes 33a and 33b, and on the proximal side (the other side in the lengthwise direction) of the centers of the two first tab through-hole 33a and 33b. Specifically, as shown in FIG. 1, the centers of the two head portion through-holes 22 formed on the distal side of the head portion 20 are located between the centers of the two first tab through-holes 33a and 33b in the width direction, and located on the proximal side of the two first tab through-holes 33a and 33b in the lengthwise direction. Accordingly, the distance between the two first-type tabs 30a and 30b can be increased, and there is a greater degree of freedom in the shape of the first tab body portions 31a and 31b. Also, workability is improved when bending the first-type tabs 30a and 30b (first connecting portions 32a and 32b).

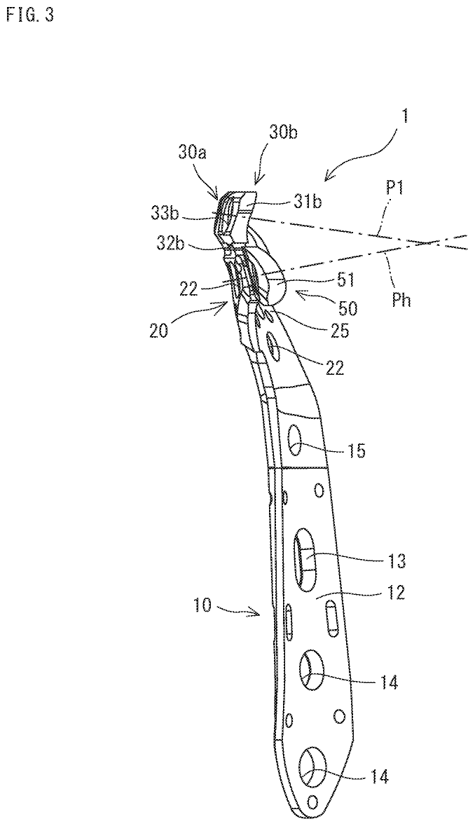

In the bone plate 1 of the present embodiment, before the first connecting portions 32a and 32b are deformed (the state shown in FIG. 1), for one of the two first-type tabs 30a and 30b (here, the first-type tab 30b provided on the right side in FIG. 1), the axis extending toward the bone of the first tab through-hole 33b passes between the axes extending toward the bone of the two head portion through-holes 22 formed on the distal (the one side in the lengthwise) of the head portion 20. In other words, when the first connecting portions 32a and 32b are in an undeformed state (the state shown in FIG. 1), the first tab through-hole of one of said two first type tabs (here, the first tab through-hole 33b) has a first tab through-hole axis (P1) extending toward the bone, and the first tab through-hole axis passes between axes extending toward the bone (Ph) of the two head portion through-holes 22 formed on the one side in the lengthwise direction in the head portion 20. Specifically, with respect to the lengthwise direction, as shown in FIG. 3, an axis extending toward the bone P1 (a first tab through-hole axis P1) of the first tab through-hole 33b is inclined to the proximal side (downward in FIG. 3), and axes extending toward the bone Ph of the two head portion through-holes 22 formed on the distal side of the head portion 20 (since the axes Ph are substantially overlapped with each other in FIG. 3, only the axis Ph on the front side is shown in FIG. 3) are inclined to the distal side (upward in FIG. 3). Furthermore, with respect to the width direction, as shown in FIG. 4, the axis extending toward the bone P1 (a first tab through-hole axis P1) of the first tab through-hole 33b is inclined leftward, and the axes extending toward the bone Ph of the two head portion through-holes 22 formed on the distal side of the head portion 20 are inclined rightward. For this reason, the axis extending toward the bone P1 (a first tab through-hole axis P1) of the first tab through-hole 33b passes between the axes extending toward the bone Ph of the two head portion through-holes 22 formed on the distal side of the head portion 20. Accordingly, even in the case where a bone screw 7 is inserted into the first tab through-hole 32b of the first-type tab 30b in accordance with the condition of the fracture (in the treatment of a fracture in the distal portion of the tibia, priority is given to inserting a bone screw 7 into a head portion through-hole 22 in the head portion 20), there is a higher possibility of being able to avoid interference with a bone screw 7 inserted into a head portion through-hole 22. Also, it is possible to use a longer bone screw 7 with the first-type tab 30b (the first tab through-hole 32b), and it is possible to expand the number of options for more appropriate treatment of fractures.

The second-type tab 40 is provided on one outside of the first-type tabs (30a, 30b) (i.e., provided in a left portion in FIG. 1). More specifically, in the present embodiment, a notch 26 is formed in a lateral portion of the distal portion of the head portion 20 (the left portion in FIG. 1), and the second-type tab 40 extends from the exposed end face on the distal side of the notch 26. Accordingly, the second-type tab 40 is provided so as not to protrude farther in the distal direction than the first-type tabs 30 (30a, 30b). The second-type tab 40 includes the bendable and twistable second connecting portion 42, and extends outward (here in the distal direction and laterally) from the head portion 20 due to the second connecting portion 42. It is preferable that the second-type tab 40 protrudes outward (laterally) farther than a lateral end portion of the head portion 20 (the left end portion in FIG. 1) in an undeformed state of the second-type tab 40. The portion of the second-type tab 40 outward of the second connecting portion 42 (the portion on the side opposite to the head portion 20) is the second tab body portion 41.

The cross-sectional area of the second connecting portion 42 is smaller than the cross-sectional area of the outer shape of the second tab body portion 41. More specifically, as shown in FIGS. 1 and 8, the second connecting portion 42 has a smaller width (w2) and a smaller thickness (t2) than the second tab body portion 41. In other words, in the second-type tab 40, the rigidity of the second connecting portion 42 is lower than that of the second tab body portion 41. Furthermore, the second connecting portion 42 has a smaller cross-sectional area than the first connecting portions 32 (32a, 32b). In the present embodiment, the width (w2) of the second connecting portion 42 is smaller than the width (w1) of the first connecting portions 32 (32a, 32b), and the thickness (t2) of the second connecting portion 42 is smaller than the thickness (t1) of the first connecting portions 32 (32a, 32b). Accordingly, in the second connecting portion 42, the second-type tab 40 is bendable toward the upper surface and the lower surface of the plate body portion and the head portion 20, and is twistable about the axis in the extending direction. Note that it is preferable that the width w2 of the second connecting portion 42 (about 2.1 to 2.3 mm in the present embodiment) is larger than the thickness t2 of the second connecting portion 42 (about 1.55 to 1.65 mm in the present embodiment), and it is preferable that the ratio of the width w2 to the thickness t2 (w2/t2) is in the range of 1.2 to 1.5. With such a range, workability is improved when deforming (in particular, twisting) the second connecting portion 42, and breakage of the second connecting portion 42 can be prevented. Also, it is preferable that the value of the ratio of the width w2 to the thickness t2 (w2/t2) of the second connecting portion 42 is smaller than the value of the ratio of the width w1 to the thickness t1 (w1/t1) of the above-described first connecting portions 32a and 32b. Accordingly, there is a clear difference between the bendable first connecting portions 32a and 32b and the bendable and twistable second connecting portion 42, and a doctor can more easily perform surgery. Note that it is preferable that the cross-sectional area of the second connecting portion 42 is set appropriately within a range smaller than the cross-sectional area of the first connecting portions. If the cross-sectional area of the second connecting portion 42 is too large, the second-type tab 40 may overall be deformed before the second connecting portion 42 is deformed (in particular, twisted), and if too small, the second connecting portion 42 may be deformed too much, and the second connecting portion 42 may become broken.

The second tab body portion 41 is located on the distal side of the second-type tab 40 (on the side toward which the second-type tab 40 extends). In the present embodiment, as shown in FIG. 1, the second tab body portion 41 of the second-type tab 40 is substantially circular. Accordingly, even in the case where the second connecting portion 42 is deformed, the outer shape of the second-type tab 40 is not likely to protrude, and contact between the bone plate 1 (including the second-type tab 40) and the skin or the like is reduced.

The second tab body portion 41 of the second-type tab 40 has the second tab through-hole 43. In the present embodiment, the second tab through-hole 43 has the same diameter as the head portion through-holes 22 (and the first tab through-holes 33a and 33b) (i.e., the diameters of the preparatory holes before formation of the female threading are the same). Also, female threading is provided on the inner surface of the second tab through-hole 43 of the second-type tab 40, and the female threading can be engaged or screwed with male threading provided on the head portion 72 of the later-described bone screw 7. Accordingly, the same type of bone screw 7 as that used with the head portion through-holes 22 (and first tab through-holes 33a and 33b) can be used with the second tab through-hole 43.

By appropriately bending and/or twisting the second connecting portion 42 of the second-type tab 40 of the bone plate 1, the bone plate 1 can be deformed more freely so as to conform to the shape of a bone (in particular, the epiphysis portion of a long bone) and the condition of the fracture, and a fracture in the end portion of the long bone can be treated more appropriately.

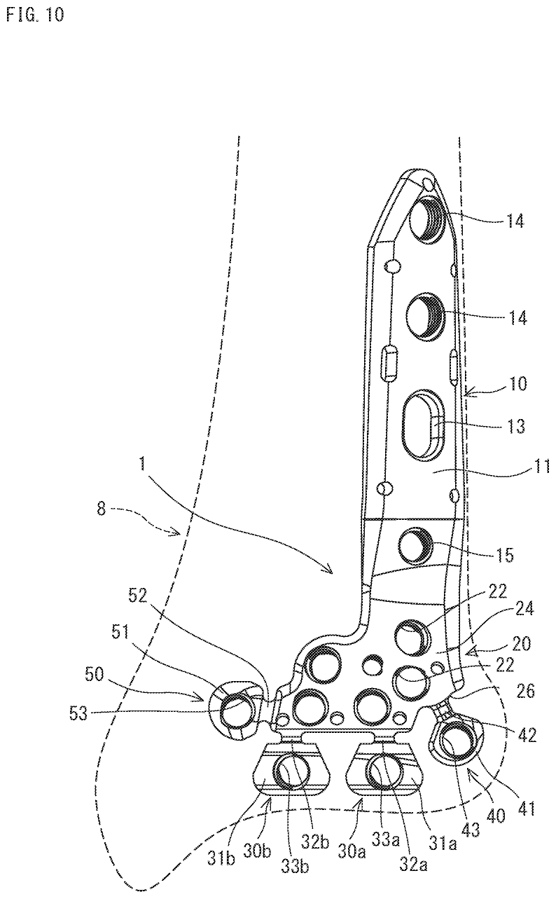

Also, as shown in FIG. 10, in the case where the bone plate 1 is used at the distal portion of the tibia 8 (indicated by dashed lines in FIG. 10), that is to say at the anterolateral face of the distal portion of the tibia (in FIG. 10, the lower side is the distal side), the second-type tab 40 is located at the anterolateral end portion (the lower right portion in FIG. 10) of the tibia 8 where the outer shape has a relatively sharp curve (small radius of curvature). Here, since the second connecting portion 42 of the second-type tab 40 is bendable and twistable, a bone fragment in the corresponding portion can be more appropriately grasped by the bone screw 7.

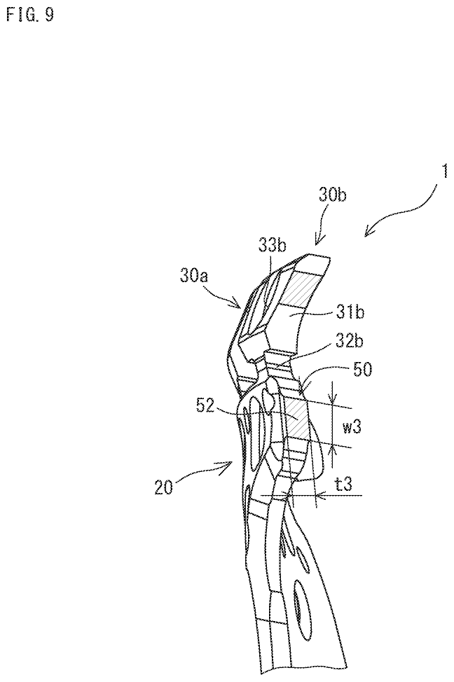

The tabs of the bone plate 1 of the present embodiment include the third-type tab 50 provided on one outside (the right portion in FIG. 1) of the first-type tabs 30 (30a, 30b), specifically the side opposite to the side on which the second-type tab 40 is provided, and the third-type tab 50 includes a third tab body portion 51 having a third tab through-hole 53, and a third connecting portion 52 that is rod-shaped or plate-shaped and connects the third tab body portion 51 and the outer peripheral portion of the head portion 20. The third-type tab 50 is provided so as to extend outward from the head portion 20 in a different direction from the first-type tabs 30, due to the third connecting portion 52, and furthermore, the minimum cross-sectional area of a widthwise cross section (the cross section shown in later-described FIG. 9) of the third connecting portion 52 is smaller than the minimum cross-sectional area of a widthwise cross section of the second connecting portion 42, and the third connecting portion 52 is bendable. The third tab through-hole 53 has the same diameter as the head portion through-holes 22.

More specifically, the third-type tab 50 is provided on one outside of the first-type tabs 30 (30a, 30b) (the right portion in FIG. 1), specifically the side of the first-type tabs 30 (30a, 30b) opposite to the side on which the second-type tab 40 is provided. In the present embodiment, the third-type tab 50 is provided so as to extend outward from the side surface (the rightward surface in FIG. 1) of the distal portion of the head portion 20. The third-type tab 50 is provided so as to extend to one side in the width direction (rightward in FIG. 1), and extends in a direction different from the first-type tabs 30 (30a, 30b) that extend in the distal direction. The third-type tab 50 includes the bendable third connecting portion 52, and extends outward from the head portion 20 due to the third connecting portion 52. The portion of the third-type tab 50 outward of the third connecting portion 52 (the portion on the side opposite to the head portion 20) is the third tab body portion 51.

The cross-sectional area of the third connecting portion 52 is smaller than the cross-sectional area of the outer shape of the third tab body portion 51. More specifically, as shown in FIGS. 1 and 9, the width (w3) of the third connecting portion 52 is smaller than that of the third tab body portion 51. In other words, in the third-type tab 50, the rigidity of the third connecting portion 52 is smaller than that of the third tab body portion 51. Accordingly, in the third connecting portion 52, the third-type tab 50 is bendable toward the upper surface and the lower surface of the plate body portion 10 and the head portion 20. Note that the thickness (t3) of the third connecting portion 52 may be smaller than the thickness of the third tab body portion 51.

Note that it is desirable that the third connecting portion 52 is bendable but not twistable (when a doctor performs an operation, the third connecting portion 52 substantially cannot be twisted about the extending direction of the third-type tab 50). For example, it is preferable that the width w3 of the third connecting portion 52 (about 2.4 to 2.6 mm in the present embodiment) is larger than the thickness t3 of the third connecting portion 52 (about 1.42 to 1.52 mm in the present embodiment), and it is preferable that the ratio of the width w3 to the thickness t3 (w3/t3) is in the range of 1.5 to 2.0. Also, it is preferable that the value of the ratio of the width w3 to the thickness t3 (w3/t3) of the third connecting portion 52 is larger than the value of the ratio of the width w2 to the thickness t2 (w2/t2) of the above-described second connecting portion 42. Accordingly, in comparison with the above-described second connecting portion 42, the third connecting portion 52 is bendable but substantially not twistable.

In the present embodiment, the third-type tab 50 is provided so as to extend to one side in the width direction (rightward in FIG. 1) from a side surface (the right side surface in FIG. 1) of the distal portion of the head portion 20, and includes the bendable third connecting portion 52. Accordingly, the shape of the head portion 20 can be set relatively freely. Specifically, by bending the third-type tab 50 to the upper surface side or the lower surface side, it is possible to avoid interference between the lateral end portion of the bone plate 1 and the bone.

The third tab body portion 51 is located on the outward side of the third-type tab 50 (on the side toward which the third-type tab 50 extends). In the present embodiment, as shown in FIG. 1, the third tab body portion 51 of the third-type tab 50 is substantially circular. Accordingly, the outer shape of the third connecting portion 52 is not likely to protrude, and contact between the bone plate 1 (including the third-type tab 50) and the skin or the like is reduced.

The third tab body portion 51 of the third-type tab 50 has the third tab through-hole 53. In the present embodiment, the third tab through-hole 53 has the same diameter as the head portion through-holes 22 (as well as the first tab through-holes 33a and 33b and the second tab through-hole 42) (i.e., the diameters of the preparatory holes before formation of the female threading are the same).

Also, female threading is provided on the inner surface of the third tab through-hole 53 of the third-type tab 50, and the female threading can be engaged or screwed with male threading provided on the head portion 72 of the later-described bone screw 7. Accordingly, the same type of bone screw 7 as that used with the head portion through-holes 22 (as well as the first tab through-holes 33a and 33b and the second tab through-hole 42) can be used with the third tab through-hole 53.

Examples of preferable materials for forming the bone plate 1 include titanium alloys (specifically, Ti-6A1-4V in JIS T 7401-2, Ti-6A1-4V ELI in ASTM F-136), pure titanium (specifically, JIS T 7401-1), and stainless steel (specifically, SUS304 and SUS316 in JIS G 4303).

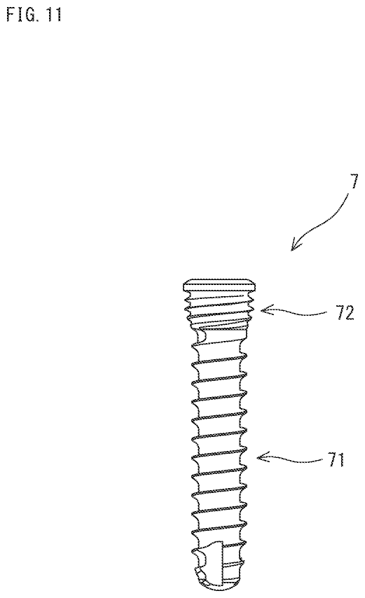

FIG. 11 shows an example of the bone screw 7 that is used together with the bone plate 1 in the treatment of a fracture in the end portion of a long bone. The bone screw 7 includes the shaft portion 71 and the head portion 72, as shown in FIG. 11. The shaft portion 71 has male threading on the outer surface, and can pass through the through-holes of the bone plate 1 (the head portion through-holes 22, the first tab through-holes 33a and 33b, the second tab through-hole 42, and the third tab through-hole 52). Also, the shaft portion 71 can enter into the bone (preparatory hole formed in the target bone).

The upper surface of the head portion 72 of the bone screw 7 includes a recession (not shown) for connecting a rotation tool (e.g., a screw driver). The recession is formed with a shape corresponding to the tip shape of the rotation tool.

The head portion 72 of the bone screw 7 has a larger diameter than the shaft portion 71, and has male threading on the outer surface. This male threading includes a helical crest portion (thread crest) that forms a so-called right-handed screw that progresses in the axial direction with clockwise rotation in plan view (in a view from the head portion 72 side in the axial direction of the bone screw 7). The male threading can be engaged or screwed with female threading formed on the inner surfaces of the through-holes of the bone plate 1 (the head portion through-holes 22, the first tab through-holes 33a and 33b, the second tab through-hole 42, and the third tab through-hole 52).

Likewise to the bone plate 1, examples of preferable materials for forming the bone screw 7 include titanium alloys (specifically, Ti-6A1-4V in JIS T 7401-2, Ti-6A1-4V ELI in ASTM F-136), pure titanium (specifically, JIS T 7401-1), and stainless steel (specifically, SUS304 and SUS316 in JIS G 4303).

The bone plate of the present invention is not limited to the embodiment described above.

For example, a configuration is possible in which a first-type tab does not have a first tab through-hole. In this case, the first-type tab (first tab body portion) has a function of appropriately supporting the fracture (bone or fracture fragment). Also, a configuration is possible in which the third-type tab is not provided, and in this case, another second-type tab may be provided on the outside of the first-type tabs opposite to the side on which the above-described second-type tab is provided in the above-described embodiment.

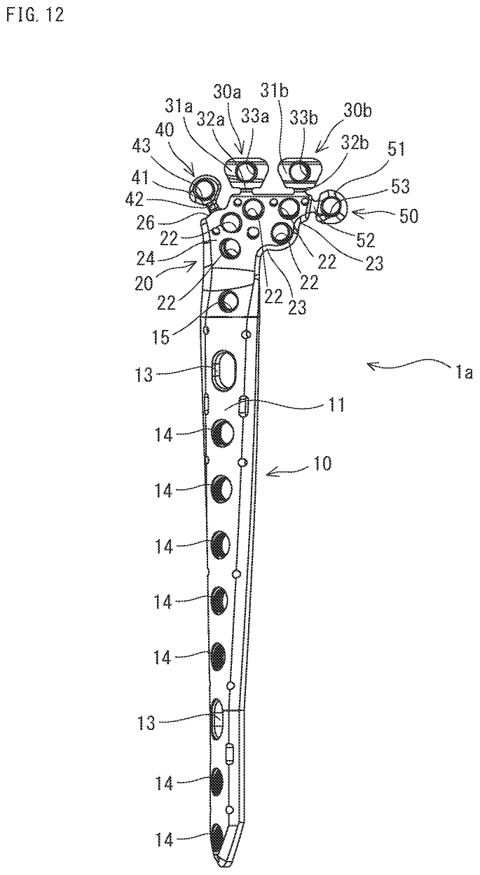

Also, the modes of the first-type tabs, the second-type tab, and the third-type tab of the bone plate (e.g., the location of formation on the head portion, the outer shapes of the first tab body portions, the second tab body portion, and the third tab body portion, and the cross-sectional shapes of the first connecting portions, the second connecting portion, and the third connecting portion), and the modes of the plate body portion and the head portion are not limited to the modes in the embodiment described above. For example, as with a bone plate 1a shown in FIG. 12, the size of the plate body portion 10 in the lengthwise may be lengthened in accordance with the shape of the long bone to be treated with the bone plate or the manner in which the bone plate is to be fixed to the long bone.

The bone plate of the present invention can be not only the bone plate illustrated in the above embodiment (an anterolateral tibia plate used for the anterolateral face of the distal portion of the tibia), but also a bone plate for use with a long bone constituting a lower limb (e.g., fibula, femur, metatarsal bone, or toe bone), or a long bone constituting an upper limb (e.g., humerus, radius, ulna, metacarpal, toe bone, or clavicle), for example. Note that the long bones to which the bone plate of the present invention is applied include a wide range of human bones that have an elongated shape (a longer length than the width).

Also, the bone plate of the present invention is used for treatment of a fracture in an end portion of a long bone, and although the end portion of the long bone to be treated by the bone plate is mainly the epiphysis of the long bone, it may also be a portion of the metaphysis or a portion of the diaphysis, in addition to the epiphyseal portion.

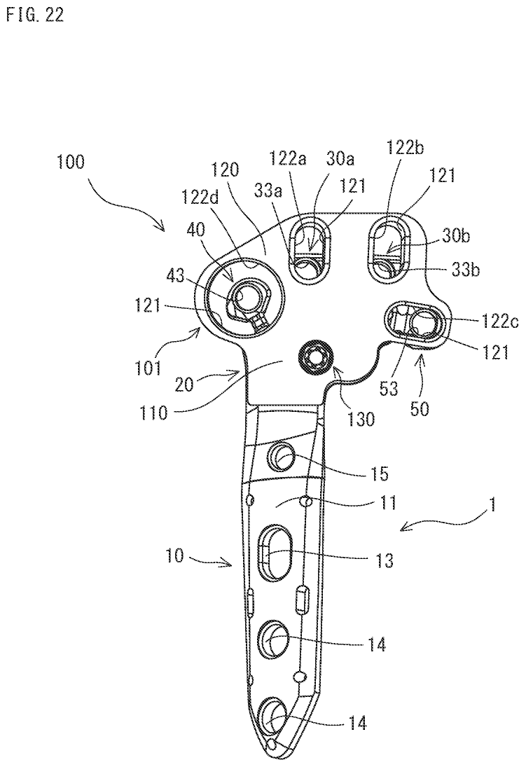

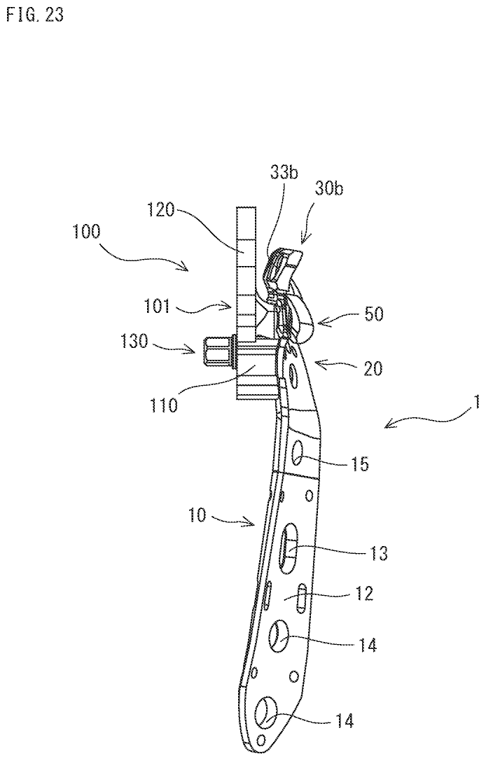

Also, as shown in FIGS. 22 and 23, a bone fracture treatment kit 200 according to the present invention includes a bone plate (e.g., the bone plate 1 described above) and a later-described deformation assisting member 100 for assisting deformation of the bone plate, and the bone plate includes deformable portions (here, the first-type tabs 30, the second-type tab 40, and the third-type tab 50) and openings (here, the first tab through-holes 33, the second tab through-hole 43, and the third tab through-hole 53) that extend through the deformable portions.

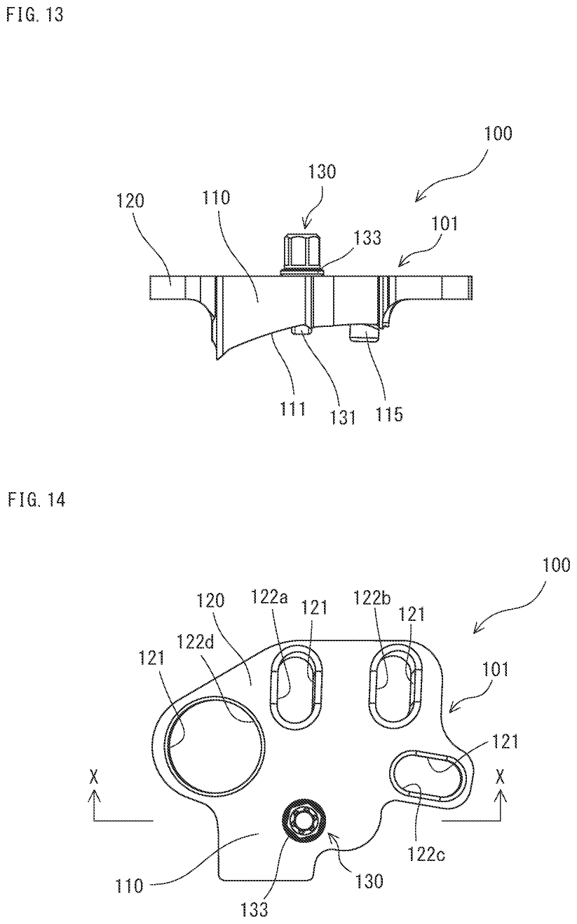

As shown in FIGS. 13 to 19, the bone plate deformation assisting member 100 of the present embodiment is for the bone plate that includes deformable portions and openings that extend through the deformable portions. The deformation assisting member 100 includes an assisting member main body portion 101 having a thickness in the vertical direction (up-down direction in FIG. 1), and the assisting member main body portion 101 includes a plate contact portion 111, which is for coming into contact with the bone plate, on the lower surface. Also, the assisting member main body portion 101 of the deformation assisting member 100 includes a plate facing portion 120 that is located a predetermined distance above the openings of the deformable portions of the bone plate when the deformation assisting member 100 is overlaid on the bone plate and the plate contact portion 111 is in contact with the bone plate, and also includes deforming tool insertion holes 122 that penetrate through the plate facing portion 120 (in other words, that extend through the assisting member main body portion 101 in the vertical direction) and each have a hard inner peripheral surface 121, and through which a rod-shaped deforming tool can be inserted. Also, the deformation assisting member 100 is configured such that the deforming tool can be inserted into any of the deforming tool insertion holes 122 and the opening of the corresponding deformable portion of the bone plate simultaneously, and the deforming tool is displaceable in the inserted state, and furthermore, the amount of deformation of the deformable portion of the bone plate is restricted by the deforming tool coming into contact with the hard inner peripheral surface 121 of the deforming tool insertion hole 122.

In the present embodiment, the plate contact portion 111 of the deformation assisting member 100 has a curved surface that covers the bone plate (the upper surface of a head portion 20 of the later-described bone plate 1).

Note that the plate contact portion is not limited to the mode described above. For example, it is not necessary that the substantially the entirety of the lower surface of the main body portion of the deformation assisting member (the portion of the assisting member main body excluding the plate facing portion) constitutes the plate contact portion for coming into contact with the bone plate, or in other words, part of the main body portion (part of the lower surface of the main body portion) may be separated from the bone plate when the plate contact portion is in contact with the bone plate. For example, the plate contact portion may be constituted by an annular rib, projections, or the like provided on the lower portion of the main body portion.

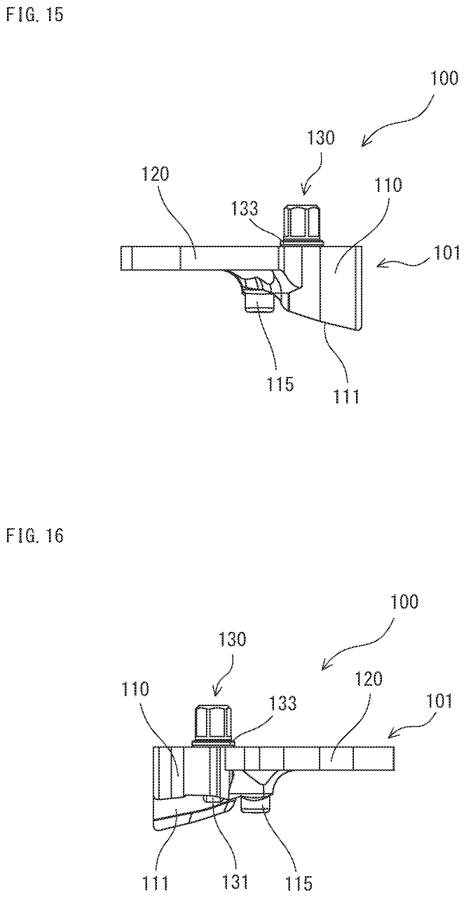

In the present embodiment, as shown in FIGS. 15 and 16, the plate facing portion 120 of the deformation assisting member 100 is a plate-shaped portion that extends laterally from the upper portion of a main body portion 110 of the deformation assisting member 100. In other words, an underfill portion (missing portion, void forming portion), which is for separating the plate facing portion 120 from the openings of the deformable portions of the bone plate (i.e., preventing contact therebetween) by a predetermined distance, is formed in the deformation assisting member 100 (assisting member main body portion 101) at a position below the plate facing portion 120. In other words, in the present embodiment, in the assisting member main body portion 101, the lower portion of the main body portion 110, which includes the plate contact portion 111, extends downward beyond the plate facing portion 120 such that the plate facing portion 120 is located a predetermined distance above the openings of the deformable portions of the bone plate when the plate contact portion 111 is in contact with the bone plate. This is to say, in the deformation assisting member 100 (assisting member main body portion 101), the main body portion 110, which includes the plate contact portion 111 and extends downward beyond the plate facing portion 120, is configured as a separating portion for positioning the plate acing portion 120 at a predetermined distance above the openings of the deformable portions of the bone plate.

As shown in FIG. 14, the deformation assisting member 100 (assisting member main body portion 101) of the present embodiment includes a plurality of (four) deforming tool insertion holes 122 (122a, 122b, 122c, and 122d).

The deforming tool insertion holes 122 (122a, 122b, 122c, 122d) include substantially oval bending insertion holes (122a, 122b, and 122c) and a substantially circular twisting insertion hole (122d). The deformation assisting member 100 includes a plurality of (here, three) bending insertion holes 122a, 122b, 122c and one twisting insertion hole 122d. Accordingly, the deformable portions of the bone plate that correspond to the deforming tool insertion holes can be appropriately deformed.

The bending insertion holes 122a, 122b, and 122c each have a substantially oval shape in plan view (shown in FIG. 14). A hard inner peripheral surface (annular inner wall surface) 121 of each of the bending insertion holes 122a, 122b, and 122c has a width in the up-down direction (the vertical direction) (up-down direction in FIG. 13), and it is preferable that the bending insertion holes 122a, 122b, and 122c allow displacement of the deforming tool in the major axis direction and prevent displacement in the minor axis direction inside the bending insertion holes 122a, 122b, and 122c. Specifically, the dimensions of the bending insertion holes 122a, 122b, and 122c in the minor axis direction are substantially the same as or slightly larger than the outer shape (outer diameter) of a later-described deforming tool 300 (tool main body portion 310), thus substantially preventing the inserted deforming tool 300 from becoming displaced in the minor axis direction. Also, the dimensions of the bending insertion holes 122a, 122b, and 122c in the major axis direction are larger than the outer shape (outer diameter) of the later-described deforming tool 300 (tool main body portion 310), thus allowing the inserted deforming tool 300 to become displaced in the major axis direction. Accordingly, the deformable portions of the bone plate that correspond to the bending insertion holes can be appropriately bent (bent in a direction set in advance). Also, in the deformation assisting member 100 of the present embodiment, due to providing the bending insertion holes in correspondence with the deformable portions of the bone plate that are formed with the intention of being bent, it is possible to prevent such deformable portions from being twisted.

The twisting insertion hole 122d has a substantially circular shape in plan view (the state shown in FIG. 14). The diameter (inner diameter) of the twisting insertion hole 122d is larger than the outer shape (outer diameter) of the later-described deforming tool 300 (tool main body portion 310), thus allowing the inserted deforming tool 300 to become displaced in any direction. Accordingly, the deformable portion of the bone plate that corresponds to the twisting insertion hole can be appropriately twisted.

The deformation assisting member 100 of the present embodiment includes a fixing threaded member accommodating hole 112 that extends through the assisting member main body portion 101 (main body portion 110) in the vertical direction, and a fixing threaded member 130 that is rotatably accommodated in the fixing threaded member accommodating hole 112 and capable of fixing the deformation assisting member 100 to the bone plate. The outer peripheral surface of the leading end portion of the fixing threaded member 130 includes a thread portion (male thread portion 131) that can be screwed into a threaded hole (later-described assisting member attachment hole 27) formed in the bone plate.

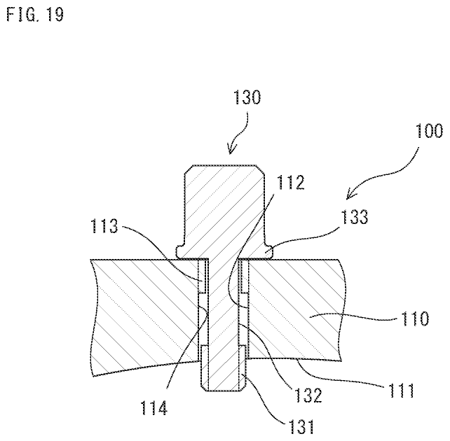

In the present embodiment, as shown in FIG. 19, a female thread portion 113, into which the male thread portion 131 of the fixing threaded member 130 can be screwed, is formed in an upper portion of the inner surface of the fixing threaded member accommodating hole 112. A portion of the fixing threaded member accommodating hole 112 below the female thread portion 113 is a threadless portion 114 where female threading is not formed. Also, a portion of the fixing threaded member 130 above (on the base side of) the male thread portion 131 is a threadless portion 132 where male threading is not formed. A flange portion 133 having an outer shape larger than the inner diameter of the fixing threaded member accommodating hole 112 extends continuously around the base end of the threadless portion 132 of the fixing threaded member 130. Accordingly, it is possible to allow the fixing threaded member 130 to be rotatably accommodated in the fixing threaded member accommodating hole 112, while preventing removal of the fixing threaded member 130 from the fixing threaded member accommodating hole 112, by interference between the fixing threaded member 130 (male thread portion 131 and flange portion 133) and the fixing threaded member accommodating hole 112 (female thread portion 113 and upper edge portion).

Also, the dimension in the vertical direction of the threadless portion 114 of the fixing threaded member accommodating hole 112 is larger than the dimension in the vertical direction of the male thread portion 131 of the fixing threaded member 130. Accordingly, by pushing the fixing threaded member 130 upward (in the fixing threaded member accommodating hole 112) from the state shown in FIG. 19 for example, the fixing threaded member 130 can be accommodated in the fixing threaded member accommodating hole 112 without the leading end (male thread portion 131) of the fixing threaded member 130 protruding from the fixing threaded member accommodating hole 112.

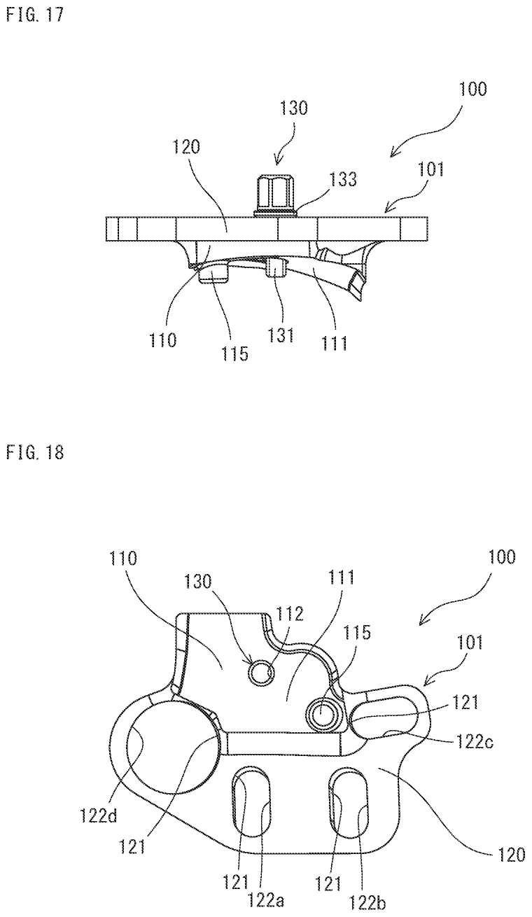

The deformation assisting member 100 includes an assisting member projecting portion 115 that projects downward from the lower surface of the assisting member main body portion 101 and can enter a recession or a hole formed in the bone plate. As shown in FIGS. 17 and 18, the assisting member projecting portion 115 is formed so as to project downward from the lower surface (plate contact portion 111) of the main body portion 110 of the assisting member main body portion 101. The assisting member projecting portion 115 has a substantially cylindrical shape, and the outer shape (outer diameter) of the assisting member projecting portion 115 is slightly smaller than the inner diameter of the recession or hole of the bone plate into which the assisting member projecting portion 115 is to enter (e.g., the head portion through-hole 22 of the bone plate 1 described above). When using the deformation assisting member 100, the assisting member projecting portion 115 is inserted into the recession or hole formed in the bone plate so as to contribute to fixing or maintaining the deformation assisting member 100 at a predetermined position and/or posture relative to the bone plate. Note that a plurality of assisting member projecting portions may be provided.

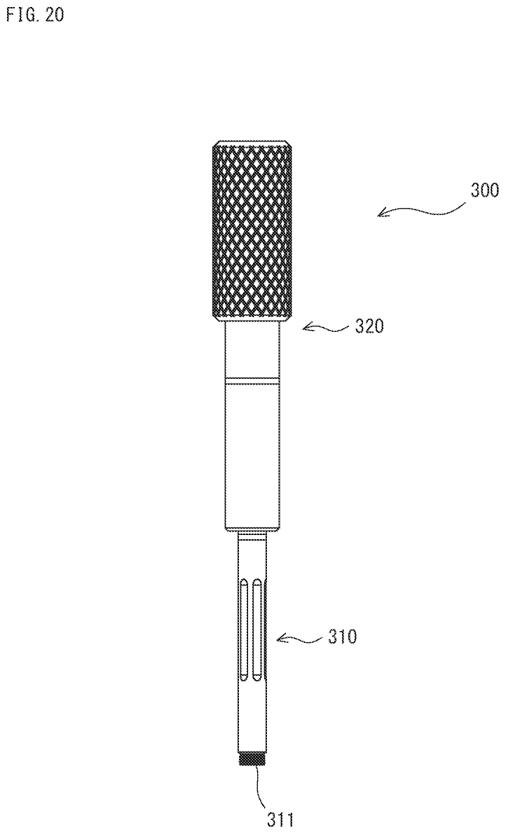

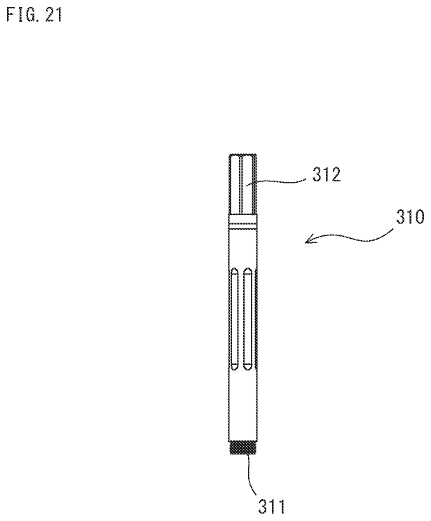

Next, a deforming tool that is suitably used with the above-described deformation assisting member will be described with use of the embodiment shown in FIGS. 20 and 21.

As shown in FIG. 20, the deforming tool 300 of the present embodiment includes a tool main body portion 310 and a grip portion 320 removably attached to the base end portion of the tool main body portion 310, and is overall shaped as a substantially straight rod (not provided with a bent portion anywhere along the full length).

The grip portion 320 can be attached to the base end portion of the tool main body portion 310. The grip portion 320 has an outer shape (outer diameter) larger than that of the tool main body portion 310, and preferably is not able to rotate relative to the tool main body portion 310 when attached to the tool main body portion 310.

As shown in FIG. 21, the tool main body portion 310 is a substantially straight rod-shaped member that has substantially the same outer shape (outer diameter) over the full length. A base end portion 312 of the tool main body portion 310 can engage with an accommodating portion provided in the leading end portion of the grip portion 320. Also, the tool main body portion 310 can be inserted into the deforming tool insertion holes 122 (122a, 122b, 122c, and 122d) of the deformation assisting member 100.

Also, the leading end portion of the deforming tool 300 (tool main body portion 310) includes a tool male thread portion 311 that can be screwed into tab female thread portions of the bone plate 1 described above (female thread portions formed in the inner surfaces of the tab through-holes 33, 43, and 53).

Next, the process of deforming the bone plate 1 with use of the above-described deformation assisting member 100 and the bone fracture treatment kit 200 including the deformation assisting member 100 will be described with use of the embodiment shown in FIGS. 22 to 25.

First, as shown in FIGS. 22 and 23, a step of attaching the deformation assisting member 100 to the bone plate 1 is performed.

Next, the deforming tool 300 is inserted into one of the deforming tool insertion holes 122 (here, the bending insertion hole 122b) and the opening of the corresponding deformable portion of the bone plate 1 (here, the first tab through-hole 33b of the first-type tab 30b). Here, the tool male thread portion 311 provided at the leading end portion of the deforming tool 300 (tool main body portion 310) is screwed into the tab female thread portion provided in the inner surface of the tab through-hole (first tab through-hole 33b).

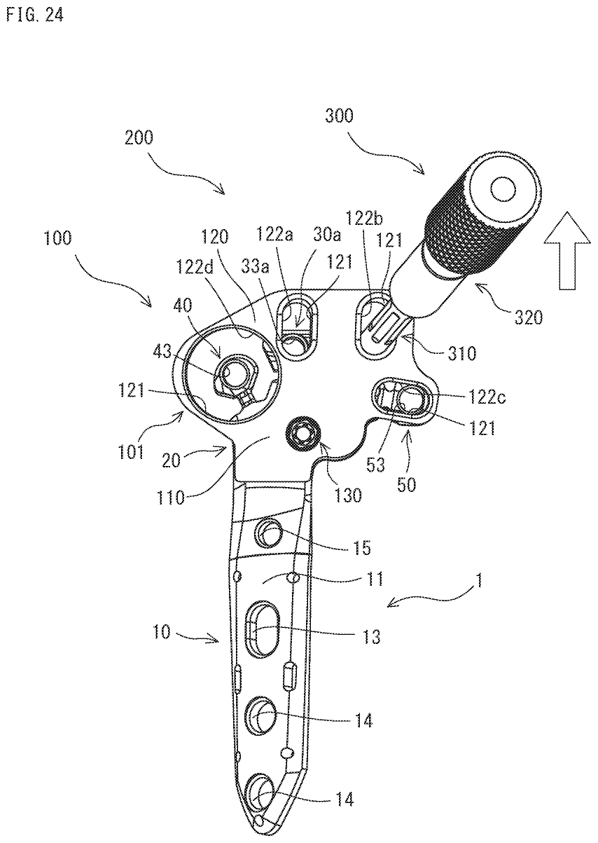

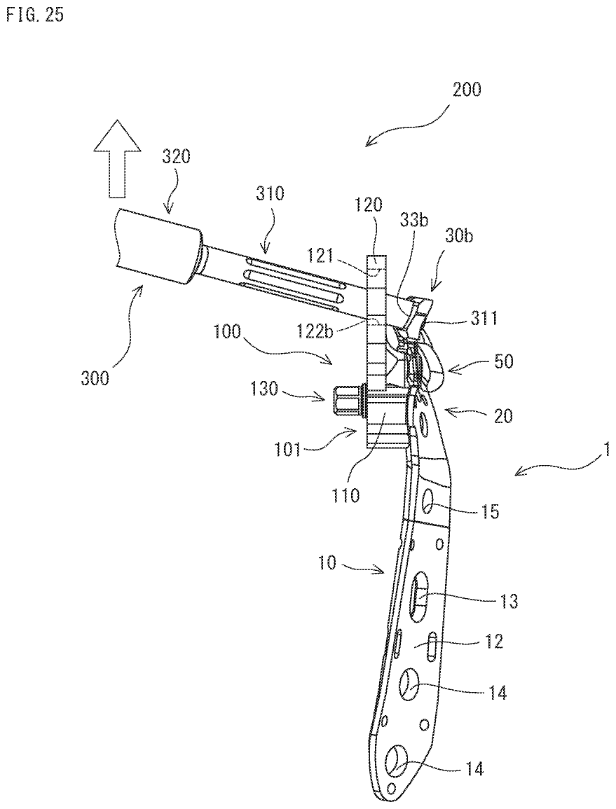

Also, in the present embodiment, as shown in FIGS. 22 to 25, in the state before deformation of the bone plate 1, the portion of the hard inner peripheral surface 121 on one side (the lower side in FIGS. 22 to 25) in the major axis direction of the deforming tool insertion hole (bending insertion hole 122b) is located in contact with or close to the outer surface of the inserted deforming tool 300. Accordingly, displacement of the deforming tool 300 to that one side in the major axis direction is restricted, and the deforming tool 300 can be guided so as to bend the deformable portion (first-type tab 30b) of the bone plate 1 in a desired direction (toward the other side in the major axis direction, which is shown by a white arrow in FIGS. 24 and 25).

Next, the grip portion 320 of the deforming tool 300 is operated (specifically, subjected to force in the direction indicated by the white arrow in

FIGS. 24 and 25) to displace the deforming tool 300, which has been inserted (or screwed) into the deforming tool insertion hole 122 (the bending insertion hole 122b) and the opening (first tab through-hole 33b). Accordingly, the deformable portion (first-type tab 30b) of the bone plate 1 undergoes deformation.

Then, the displacement of the deforming tool 300 (deformation of the deformable portion of the bone plate 1) continues, and at a certain point, the deforming tool 300 and the hard inner peripheral surface 121 of the deforming tool insertion hole 122 come into contact, thus restricting further displacement of the deforming tool 300. Accordingly, the amount of deformation of the bone plate 1 can be restricted to a desired amount, and damage to the bone plate 1 can be prevented.

The following is an aspect of a bone plate of the present invention.

(1) A bone plate for use in treatment of a fracture in an end portion of a long bone, comprising:

a plate body portion extending in a lengthwise direction;

a head portion extending from one end portion in the lengthwise direction of the plate body portion and having a larger width than the one end portion; and

tabs on an outer side of an outer peripheral portion of the head portion, wherein

the head portion has a plurality of head portion through-holes into which a shaft portion of a bone screw is insertable,

the tabs comprise first-type tabs side by side with each other on the outer side of the outer peripheral portion of the head portion and a second-type tab on one outside of the first-type tabs,

each of the first-type tabs includes:
a first tab body portion; and
a first connecting portion that is rod-shaped or plate-shaped and connects the first tab body portion and the outer peripheral portion of the head portion,

a cross-sectional area of a widthwise cross section of the first connecting portion is smaller than a cross-sectional area of a widthwise cross section of an outer shape of the first tab body portion,

the first connecting portion is bendable,

the second-type tab includes:
a second tab body portion having a second tab through-hole; and
a second connecting portion that is rod-shaped or plate-shaped and connects the second tab body portion and the outer peripheral portion of the head portion,

a minimum cross-sectional area of a widthwise cross section of the second connecting portion is smaller than a minimum cross-sectional area of a widthwise cross section of the first connecting portion, and

the second connecting portion is bendable and twistable, and

wherein the bone plate is for use in treatment of a fracture in an end portion of a long bone.

A bone plate according to the present invention includes: a plate body portion extending in a lengthwise direction; a head portion extending from one end portion in the lengthwise direction of the plate body portion and having a larger width than the one end portion; and tabs on an outer side of an outer peripheral portion of the head portion, wherein the tabs comprise first-type tabs side by side with each other on the outer side of the outer peripheral portion of the head portion; and a second-type tab on one outside of the first-type tabs, each of the first-type tabs includes: a first tab body portion; and a first connecting portion that is rod-shaped or plate-shaped and connects the first tab body portion and the outer peripheral portion of the head portion, a cross-sectional area of a widthwise cross section of the first connecting portion is smaller than a cross-sectional area of a widthwise cross section of an outer shape of the first tab body portion, the first connecting portion is bendable, the second-type tab includes: a second tab body portion having a second tab through-hole; and a second connecting portion that is rod-shaped or plate-shaped and connects the second tab body portion and the outer peripheral portion of the head portion, a minimum cross-sectional area of a widthwise cross section of the second connecting portion is smaller than a minimum cross-sectional area of a widthwise cross section of the first connecting portion, and the second connecting portion is bendable and twistable. According to this configuration, by appropriately bending the first connecting portions of the first-type tab and appropriately bending and/or twisting the second connecting portion of the second-type tab, the bone plate can be deformed more freely so as to conform to the shape of a bone and the condition of a fracture, and a fracture in an end portion of a long bone can be treated more appropriately.

The above-described aspect may be configured as described below.

(2) The bone plate according to above (1), wherein

a value of a ratio of a width w2 to a thickness t2 (w2/t2) of a minimum cross-sectional portion of the widthwise cross section of the second connecting portion is smaller than a value of a ratio of a width w1 to a thickness t1 (w1/t1) of a minimum cross-sectional portion of the widthwise cross section of the first connecting portion.

(3) The bone plate according to above (2), wherein

the value of the ratio of the width w2 to the thickness t2 (w2/t2) of the minimum cross-sectional portion of the widthwise cross section of the second connecting portion is in a range of 1.2 to 1.5, and

the value of the ratio of the width w1 to the thickness t1 (w1/t1) of the minimum cross-sectional portion of the widthwise cross section of the first connecting portion is in a range of 1.5 to 2.0.

(4) The bone plate according to any one of above (1) to (3), wherein

the second-type tab does not protrude farther in a distal direction than the first-type tabs, and

the second-type tab protrudes outward (laterally) farther than a lateral end portion of the head portion, in an undeformed state of the second-type tab.

(5) The bone plate according to any one of above (1) to (4), wherein

the first tab body portion of each of the first-type tabs has a first tab through-hole, and

the first tab through-holes and the second tab through-hole have a diameter equivalent to a diameter of the head portion through-holes.

(6) The bone plate according to any one of above (1) to (5), wherein

the tabs further include a third-type tab provided on an outside of the first-type tabs opposite to a side on which the second-type tab is provided,

the third-type tab includes:
a third tab body portion having a third tab through-hole; and
a third connecting portion that is rod-shaped or plate-shaped and connects the third tab body portion and the outer peripheral portion of the head portion,

due to the third connecting portion, the third-type tab extends outward from the head portion in a different direction from the first-type tabs,

a minimum cross-sectional area of a widthwise cross section of the third connecting portion is larger than the minimum cross-sectional area of the widthwise cross section of the second connecting portion,

the third connecting portion is bendable, and

the third tab through-hole has a diameter equivalent to a diameter of the head portion through-holes.

(7) The bone plate according to above (6), wherein

a ratio of a width w3 to a thickness t3 (w3/t3) of a cross section of the third connecting portion along a plane orthogonal to an extending direction of the third-type tab is in a range of 1.5 to 2.0, and is larger than a value of a ratio of a width w2 to a thickness t2 (w2/t2) of a cross section of the second connecting portion along a plane orthogonal to an extending direction of the second-type tab.

(8) The bone plate according to any one of above (1) to (7), wherein

the tabs include two of the first-type tabs,

the first tab body portion of each of the two first-type tabs has a first tab through-hole, and

centers of two head portion through-holes formed on one side in the lengthwise direction in the head portion are located between centers of the two first tab through-holes with respect to a width direction of the head portion, and on another side in the lengthwise direction of the centers of the two first tab through-holes.

(9) The bone plate according to above (8), wherein

when the first connecting portion is in an undeformed state, the first tab through-hole of one of said two first-type tabs has a first tab through-hole axis extending toward the bone, and the first tab through-hole axis passes between axes extending toward the bone of the two head portion through-holes formed on the one side in the lengthwise direction in the head portion.

(10) The bone plate according to any one of above (1) to (9), wherein

the first tab body portion of each of the first-type tabs is substantially shaped as a trapezoid that widens toward one side in the lengthwise direction.

(11) The bone plate according to above (10), wherein

in each of the first-type tabs, a maximum dimension in a width direction of the first tab body portion is 1.5 or more times a minimum dimension of the first tab body portion.

(12) The bone plate according to any one of above (1) to (11), wherein

the second tab body portion of the second-type tab is substantially circular.

(13) The bone plate according to above (6) or (7), wherein

the first tab body portion of each of the first-type tabs is substantially shaped as a trapezoid that widens toward one side in the lengthwise direction, and

the second tab body portion of the second-type tab and the third tab body portion of the third-type tab are each substantially circular.

(14) The bone plate according to any one of above (1) to (13), wherein

the first tab body portion of each of the first-type tabs has a first tab through-hole, and

in each of the first-type tabs, in a projected area of the first tab through-hole as viewed from an axial direction, a projected area of an outer shape of the first tab body portion is 4 times or more the projected area of the first tab through-hole.

(15) The bone plate according to any one of above (1) to (14), wherein

the bone plate is for use in treatment of a fracture in a distal portion of a tibia.

The following is another aspect of a bone plate of the present invention.

(16) A bone plate for use in treatment of a fracture in an end portion of a long bone, comprising:

a plate body portion extending in a lengthwise direction;

a head portion extending from one end portion in the lengthwise direction of the plate body portion and having a larger width than the one end portion; and

tabs on an outer side of an outer peripheral portion of the head portion, wherein

the tabs comprise first-type tabs side by side with each other on the outer side of the outer peripheral portion of the head portion, a second-type tab on one outside of the first-type tabs; and a third-type tab on an outside of the first-type tabs opposite to a side on which the second-type tab is provided,

each of the first-type tabs includes:
a first tab body portion; and
a first connecting portion that is rod-shaped or plate-shaped and connects the first tab body portion and the outer peripheral portion of the head portion,

a cross-sectional area of a widthwise cross section of the first connecting portion is smaller than a cross-sectional area of a widthwise cross section of an outer shape of the first tab body portion,

the first connecting portion is bendable,

the second-type tab includes:
a second tab body portion having a second tab through-hole; and
a second connecting portion that is rod-shaped or plate-shaped and connects the second tab body portion and the outer peripheral portion of the head portion,

a minimum cross-sectional area of a widthwise cross section of the second connecting portion is smaller than a minimum cross-sectional area of a widthwise cross section of the first connecting portion,

the second connecting portion is bendable and twistable,

the third-type tab includes:
a third tab body portion having a third tab through-hole; and
a third connecting portion that is rod-shaped or plate-shaped and connects the third tab body portion and the outer peripheral portion of the head portion,

due to the third connecting portion, the third-type tab extends outward from the head portion in a different direction from the first-type tabs,

a minimum cross-sectional area of a widthwise cross section of the third connecting portion is larger than the minimum cross-sectional area of the widthwise cross section of the second connecting portion, and

the third connecting portion is bendable.

The above-described aspect may be configured as described below.

(17) The bone plate according to above (16), wherein

the first tab body portion of each of the first-type tabs is substantially shaped as a trapezoid that widens toward one side in the lengthwise direction, and

the second tab body portion of the second-type tab and the third tab body portion of the third-type tab are each substantially circular.

(18) The bone plate according to above (16) or (17), wherein

the head portion has a plurality of head portion through-holes into which a shaft portion of a bone screw is insertable,

the first tab body portion of each of the first-type tabs has a first tab through-hole, and

the first tab through-holes, the second tab through-hole, and the third tab through-hole each have a diameter equivalent to a diameter of the head portion through-holes.

The following is an aspect of a bone fracture treatment kit of the present invention.

(19) A bone fracture treatment kit comprising:

the bone plate according to any one of above (1) to (18) and a deformation assisting member for the bone plate,

wherein the bone plate includes deformable portions having an opening extending through thereof,

the deformable portions include the first-type tabs and/or the second-type tab,

the openings form first tab through-holes provided in the first tab body portions of the first-type tabs and/or the second tab through-hole of the second-type tab,

the deformation assisting member includes an assisting member main body having a thickness in a vertical direction,

the assisting member main body has a lower surface including a plate contact portion configured to come into contact with the bone plate,

the assisting member main body includes:
a plate facing portion configured to be located a predetermined distance above the openings of the deformable portion of the bone plate when the deformation assisting member is overlaid on the bone plate and the plate contact portion is in contact with the bone plate; and
a deforming tool insertion hole that penetrates through the plate facing portion and has a hard inner peripheral surface, and through which a rod-shaped deforming tool is insertable,

the deformation assisting member is further configured such that the deforming tool can be inserted into the deforming tool insertion hole and the opening of the deformable portion of the bone plate simultaneously, and the deforming tool is displaceable in the inserted state, and

an amount of deformation of the deformable portion of the bone plate is restricted by the deforming tool coming into contact with the hard inner peripheral surface of the deforming tool insertion hole.

What is claimed is:

1. A bone plate for use in treatment of a fracture in an end portion of a long bone, comprising:
a plate body portion extending in a lengthwise direction;
a head portion extending from one end portion in the lengthwise direction of the plate body portion and having a larger width than the one end portion; and
tabs on an outer side of an outer peripheral portion of the head portion, wherein
the head portion has a plurality of head portion through-holes into which a shaft portion of a bone screw is insertable,
the tabs comprise first-type tabs side by side with each other on the outer side of the outer peripheral portion of the head portion and a second-type tab on one outside of the first-type tabs,
each of the first-type tabs includes:
a first tab body portion; and
a first connecting portion that is rod-shaped or plate-shaped and connects the first tab body portion and the outer peripheral portion of the head portion,
a cross-sectional area of a widthwise cross section of the first connecting portion is smaller than a cross-sectional area of a widthwise cross section of an outer shape of the first tab body portion,
the first connecting portion is bendable,
the second-type tab includes:
a second tab body portion having a second tab through-hole; and
a second connecting portion that is rod-shaped or plate-shaped and connects the second tab body portion and the outer peripheral portion of the head portion,
a minimum cross-sectional area of a widthwise cross section of the second connecting portion is smaller than a minimum cross-sectional area of a widthwise cross section of the first connecting portion, and
the second connecting portion is bendable and twistable, and
wherein the bone plate is for use in treatment of a fracture in an end portion of a long bone.

2. The bone plate according to claim 1, wherein
a value of a ratio of a width w2 to a thickness t2 (w2/t2) of a minimum cross-sectional portion of the widthwise cross section of the second connecting portion is smaller than a value of a ratio of a width w1 to a thickness t1 (w1/t1) of a minimum cross-sectional portion of the widthwise cross section of the first connecting portion.

3. The bone plate according to claim 2, wherein
the value of the ratio of the width w2 to the thickness t2 (w2/t2) of the minimum cross-sectional portion of the widthwise cross section of the second connecting portion is in a range of 1.2 to 1.5, and
the value of the ratio of the width w1 to the thickness t1 (w1/t1) of the minimum cross-sectional portion of the widthwise cross section of the first connecting portion is in a range of 1.5 to 2.0.

4. The bone plate according to claim 1, wherein
the second-type tab does not protrude farther in a distal direction than the first-type tabs, and
the second-type tab protrudes outward (laterally) farther than a lateral end portion of the head portion, in an undeformed state of the second-type tab.

5. The bone plate according to claim 1, wherein
the first tab body portion of each of the first-type tabs has a first tab through-hole, and
the first tab through-holes and the second tab through-hole have a diameter equivalent to a diameter of the head portion through-holes.

6. The bone plate according to claim 1, wherein
the tabs further include a third-type tab provided on an outside of the first-type tabs opposite to a side on which the second-type tab is provided,
the third-type tab includes:
a third tab body portion having a third tab through-hole; and
a third connecting portion that is rod-shaped or plate-shaped and connects the third tab body portion and the outer peripheral portion of the head portion,
due to the third connecting portion, the third-type tab extends outward from the head portion in a different direction from the first-type tabs,
a minimum cross-sectional area of a widthwise cross section of the third connecting portion is larger than the minimum cross-sectional area of the widthwise cross section of the second connecting portion,
the third connecting portion is bendable, and
the third tab through-hole has a diameter equivalent to a diameter of the head portion through-holes.

7. The bone plate according to claim 6, wherein
a ratio of a width w3 to a thickness t3 (w3/t3) of a cross section of the third connecting portion along a plane orthogonal to an extending direction of the third-type tab is in a range of 1.5 to 2.0, and is larger than a value of a ratio of a width w2 to a thickness t2 (w2/t2) of a cross section of the second connecting portion along a plane orthogonal to an extending direction of the second-type tab.

8. The bone plate according to claim 1, wherein
the tabs include two of the first-type tabs,
the first tab body portion of each of the two first-type tabs has a first tab through-hole, and
centers of two head portion through-holes formed on one side in the lengthwise direction in the head portion are located between centers of the two first tab through-holes with respect to a width direction of the head portion, and on another side in the lengthwise direction of the centers of the two first tab through-holes.

9. The bone plate according to claim 8, wherein
when the first connecting portion is in an undeformed state, the first tab through-hole of one of said two first-type tabs has a first tab through-hole axis extending toward the bone, and the first tab through-hole axis passes between axes extending toward the bone of the two head portion through-holes formed on the one side in the lengthwise direction in the head portion.

10. The bone plate according to claim 1, wherein
the first tab body portion of each of the first-type tabs is substantially shaped as a trapezoid that widens toward one side in the lengthwise direction.

11. The bone plate according to claim 10, wherein
in each of the first-type tabs, a maximum dimension in a width direction of the first tab body portion is 1.5 or more times a minimum dimension of the first tab body portion.

12. The bone plate according to claim 10, wherein
the second tab body portion of the second-type tab is substantially circular.

13. The bone plate according to claim 6, wherein
the first tab body portion of each of the first-type tabs is substantially shaped as a trapezoid that widens toward one side in the lengthwise direction, and
the second tab body portion of the second-type tab and the third tab body portion of the third-type tab are each substantially circular.

14. The bone plate according to claim 1, wherein
the first tab body portion of each of the first-type tabs has a first tab through-hole, and
in each of the first-type tabs, in a projected area of the first tab through-hole as viewed from an axial direction, a projected area of an outer shape of the first tab body portion is 4 times or more the projected area of the first tab through-hole.

15. The bone plate according to claim 1, wherein
the bone plate is for use in treatment of a fracture in a distal portion of a tibia.

16. A bone plate for use in treatment of a fracture in an end portion of a long bone, comprising:
a plate body portion extending in a lengthwise direction;
a head portion extending from one end portion in the lengthwise direction of the plate body portion and having a larger width than the one end portion; and
tabs on an outer side of an outer peripheral portion of the head portion, wherein
the tabs comprise first-type tabs side by side with each other on the outer side of the outer peripheral portion of the head portion, a second-type tab on one outside of the first-type tabs; and a third-type tab on an outside of the first-type tabs opposite to a side on which the second-type tab is provided,
each of the first-type tabs includes:
a first tab body portion; and
a first connecting portion that is rod-shaped or plate-shaped and connects the first tab body portion and the outer peripheral portion of the head portion,
a cross-sectional area of a widthwise cross section of the first connecting portion is smaller than a cross-sectional area of a widthwise cross section of an outer shape of the first tab body portion,
the first connecting portion is bendable,
the second-type tab includes:
a second tab body portion having a second tab through-hole; and
a second connecting portion that is rod-shaped or plate-shaped and connects the second tab body portion and the outer peripheral portion of the head portion,
a minimum cross-sectional area of a widthwise cross section of the second connecting portion is smaller than a minimum cross-sectional area of a widthwise cross section of the first connecting portion,
the second connecting portion is bendable and twistable,
the third-type tab includes:
a third tab body portion having a third tab through-hole; and
a third connecting portion that is rod-shaped or plate-shaped and connects the third tab body portion and the outer peripheral portion of the head portion,
due to the third connecting portion, the third-type tab extends outward from the head portion in a different direction from the first-type tabs,
a minimum cross-sectional area of a widthwise cross section of the third connecting portion is larger than the minimum cross-sectional area of the widthwise cross section of the second connecting portion, and
the third connecting portion is bendable.

17. The bone plate according to claim 16, wherein
the first tab body portion of each of the first-type tabs is substantially shaped as a trapezoid that widens toward one side in the lengthwise direction, and
the second tab body portion of the second-type tab and the third tab body portion of the third-type tab are each substantially circular.

18. The bone plate according to claim 16, wherein
the head portion has a plurality of head portion through-holes into which a shaft portion of a bone screw is insertable,
the first tab body portion of each of the first-type tabs has a first tab through-hole, and
the first tab through-holes, the second tab through-hole, and the third tab through-hole each have a diameter equivalent to a diameter of the head portion through-holes.

19. A bone fracture treatment kit comprising:
the bone plate according to claim 1; and
a deformation assisting member for the bone plate,
wherein the bone plate includes deformable portions having an opening extending through thereof,
the deformable portions include the first-type tabs and/or the second-type tab,
the openings form first tab through-holes provided in the first tab body portions of the first-type tabs and/or the second tab through-hole of the second-type tab,
the deformation assisting member includes an assisting member main body having a thickness in a vertical direction,
the assisting member main body has a lower surface including a plate contact portion configured to come into contact with the bone plate,
the assisting member main body includes:
a plate facing portion configured to be located a predetermined distance above the openings of the deformable portion of the bone plate when the deformation assisting member is overlaid on the bone plate and the plate contact portion is in contact with the bone plate; and
a deforming tool insertion hole that penetrates through the plate facing portion and has a hard inner peripheral surface, and through which a rod-shaped deforming tool is insertable,
the deformation assisting member is further configured such that the deforming tool can be inserted into the deforming tool insertion hole and the opening of the deformable portion of the bone plate simultaneously, and the deforming tool is displaceable in the inserted state, and
an amount of deformation of the deformable portion of the bone plate is restricted by the deforming tool coming into contact with the hard inner peripheral surface of the deforming tool insertion hole.
